(19) United States
(12) Patent Application Publication
(10) Pub. No.: US 2024/0389951 A1
(43) Pub. Date: Nov. 28, 2024

(54) NON-CONTACT DETECTION DEVICE AND DETECTION METHOD FOR ELECTROCARDIOGRAM SIGNAL

(71) Applicant: Streamteck Scientific Inc., Taipei City (TW)

(72) Inventor: Yu-Hsiang Su, New Taipei City (TW)

(73) Assignee: Streamteck Scientific Inc., Taipei City (TW)

(21) Appl. No.: 18/664,266

(22) Filed: May 14, 2024

(30) Foreign Application Priority Data
May 26, 2023 (TW) 112119699

(51) Int. Cl. A61B 5/00; A61B 5/352; A61B 5/353; A61B 5/355

(52) U.S. Cl. CPC A61B 5/7278; A61B 5/352; A61B 5/353; A61B 5/355; A61B 5/7228

(57) ABSTRACT

A non-contact detection device and detection method for an electrocardiogram (ECG) signal are provided. The detection method includes: transmitting a first wireless signal and receiving a first reflected signal corresponding to the first wireless signal; pre-processing the first reflected signal to generate a first processed signal; capturing a first embedding from the first processed signal; generating an estimated ECG signal according to the first embedding; and outputting the estimated ECG signal.

### CROSS-REFERENCE TO RELATED APPLICATION

This application claims the priority benefit of Taiwan application serial no. 112119699, filed on May 26, 2023. The entirety of the above-mentioned patent application is hereby incorporated by reference herein and made a part of specification.

### BACKGROUND

### Technical Field

The disclosure relates to an electrocardiogram (ECG) signal detection technology, and in particular, to a non-contact detection device and detection method for an ECG signal.

### Description of Related Art

ECG signals can be used to record the electrophysiological activity of the heart. Doctors can diagnose the subject based on the ECG signals. Currently, the contact ECG signal measurement method that is clinically used relies on electrode patches. When measuring ECG signals, medical personnel need to stick several electrode patches to specific parts of the subject's body. The process of sticking electrode patches is cumbersome and may easily increase the workload of medical personnel or cause discomfort to the subject, and may even increase the risk of disease infection for medical personnel. Therefore, how to improve the subject's comfort during ECG signal measurement and increase the efficiency of ECG signal measurement is one of the important issues in the art.

### SUMMARY

The disclosure provides a non-contact detection device and detection method for an electrocardiogram (ECG) signal capable of measuring an ECG signal of a subject through wireless signals.

The disclosure provides a non-contact detection device for an ECG signal including a first transceiver, a storage medium, and a processor. The storage medium stores a plurality of modules. The processor is coupled to the storage medium and the first transceiver and accesses and executes the plurality of modules. The plurality of modules include a communication module, a pre-processing module, a wireless signal encoder, a decoder, and a computing module. The communication module transmits a first wireless signal through the first transceiver and receives a first reflected signal corresponding to the first wireless signal. The pre-processing module pre-processes the first reflected signal to generate a first processed signal. The wireless signal encoder captures a first embedding from the first reflected signal and the first processed signal. The decoder generates an estimated ECG signal according to the first embedding. The computing module outputs the estimated ECG signal.

In an embodiment of the disclosure, the detection device further includes a second transceiver. The second transceiver is coupled to the processor and detects a first ECG signal. The plurality of modules further include an ECG signal encoder. The ECG signal encoder captures a second embedding from the first ECG signal. The communication module transmits a second wireless signal through the first transceiver and receives a second reflected signal corresponding to the second wireless signal. The pre-processing module pre-processes the second reflected signal to generate a second processed signal. The computing module trains the wireless signal encoder according to the second reflected signal and the second processed signal based on a first machine learning algorithm. A first loss function of the first machine learning algorithm is associated with the second embedding.

In an embodiment of the disclosure, the communication module transmits a third wireless signal through the first transceiver and receives a third reflected signal corresponding to the third wireless signal. The communication module detects a second ECG signal through the second transceiver. The computing module trains the pre-processing module according to the third reflected signal based on a second machine learning algorithm. A second loss function of the second machine learning algorithm is associated with the second ECG signal.

In an embodiment of the disclosure, the computing module detects a time when a waveform appears in the second ECG signal, and label data used to train the pre-processing module includes the time.

In an embodiment of the disclosure, the waveform includes at least one of a P wave, a Q wave, a R wave, a S wave, and a T wave.

In an embodiment of the disclosure, the detection device further includes a second transceiver. The second transceiver is coupled to the processor and detects a third ECG signal. The plurality of modules further include an ECG signal encoder. The ECG signal encoder captures a third embedding from the third ECG signal. The computing module trains or updates the ECG signal encoder and the decoder according to the third ECG signal and the third embedding based on a third machine learning algorithm. A third loss function of the third machine learning algorithm is associated with the third ECG signal.

In an embodiment of the disclosure, the third loss function is associated with a spectrum of the third ECG signal.

In an embodiment of the disclosure, at least one of the pre-processing module, the wireless signal encoder, the decoder, and the ECG signal encoder is a transformer model.

In an embodiment of the disclosure, the first wireless signal includes a frequency modulated continuous wave signal carried by millimeter waves.

The disclosure further provides a non-contact detection method for an electrocardiogram (ECG) signal, and the method includes the following steps. A first wireless signal is transmitted, and a first reflected signal corresponding to the first wireless signal is received. The first reflected signal is pre-processed to generate a first processed signal. A first embedding is captured from the first reflected signal and the first processed signal. An estimated ECG signal is generated according to the first embedding. The estimated ECG signal is output.

To sum up, the detection device provided by the disclosure is able to obtain the ECG signal of the subject in a non-contact manner. In the disclosure, the quality of clinical medical care is improved, the comfort of subjects is enhanced, and the workload of medical personnel or the risk of disease infection is lowered.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic view illustrating a non-contact detection device for an electrocardiogram (ECG) signal according to an embodiment of the disclosure.

FIG. 2 is a schematic diagram illustrating estimation of an ECG signal through wireless signal measurement according to an embodiment of the disclosure.

FIG. 3 is a schematic diagram illustrating training of a wireless signal encoder according to an embodiment of the disclosure.

FIG. 4 is a schematic diagram illustrating training of a pre-processing module according to an embodiment of the disclosure.

FIG. 5 is a schematic diagram illustrating training of a decoder and an ECG signal encoder according to an embodiment of the disclosure.

FIG. 6 is a flow chart illustrating a non-contact detection method for an ECG signal according to an embodiment of the disclosure.

### DESCRIPTION OF THE EMBODIMENTS

In order to make the disclosure more comprehensible, several embodiments are described below as examples of implementation of the disclosure. Moreover, elements/components/steps with the same reference numerals are used to represent the same or similar parts in the drawings and embodiments.

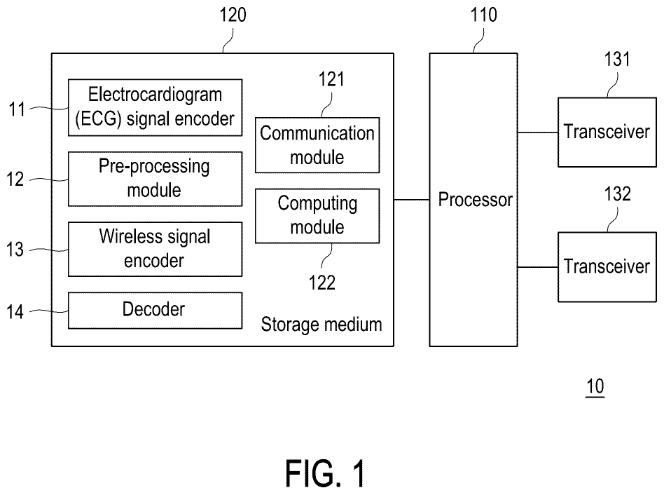

FIG. 1 is a schematic view illustrating a non-contact detection device 10 for an electrocardiogram (ECG) signal according to an embodiment of the disclosure. The detection device 10 may include a processor 110, a storage medium 120, a transceiver 131, and a transceiver 132.

The processor 110 may be, for example, a central processing unit (CPU), a programmable micro control unit (MCU) for general or special use, a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA), other similar devices, or a combination of the foregoing devices. The processor 110 may be coupled to the storage medium 120, the transceiver 131, and the transceiver 132, and access and execute a plurality of modules and various applications stored in the storage medium 120.

The storage medium 120 is, for example, a fixed or movable random access memory (RAM) in any form, a read-only memory (ROM), a flash memory, a hard disk drive (HDD), a solid state drive (SSD), a similar device, or a combination of the foregoing devices, and is used to store the plurality of modules or various applications that can be executed by the processor 110. In this embodiment, the storage medium 120 may store a plurality of modules or models including a communication module 121, a computing module 122, an ECG signal encoder 11, a pre-processing module 12, a wireless signal encoder 13, and a decoder 14, and functions of these modules or models are to be described in the following paragraphs. The ECG signal encoder 11, the pre-processing module 12, the wireless signal encoder 13, or the decoder 14 may be implemented by a machine learning model such as a deep learning model or a transformer model, but the disclosure is not limited thereto.

The transceiver 131 or the transceiver 132 is used for transmitting and receiving signals. The transceiver 131 or the transceiver 132 may also perform, for example, low noise amplification, impedance matching, mixing, up or down frequency conversion, filtering, amplification, and similar operations. In an embodiment, the processor 110 may receive a signal from or transmit a signal to an external electronic device through the transceiver 131 or the transceiver 132.

The communication module 121 may transmit a wireless signal to a subject through the transceiver 131 and receive a reflected signal corresponding to the wireless signal. The reflected signal may contain information related to micro-vibrations in the subject's chest. The reflected signal may be used to generate an estimated ECG signal. The signal sent by the transceiver 131 is, for example, a frequency modulated continuous wave (FMCW) signal carried by millimeter waves (mmWave).

The communication module 121 may measure an ECG signal of the subject through the transceiver 132. To be specific, the transceiver 132 may be coupled to one or more electrodes attached to the subject and obtains the ECG signal through the one or more electrodes. The ECG signal may include any one or a combination of the 12 sets of lead signals. According to the user's needs, each module or model in the detection device 10 may be trained to restore the reflected signal to any one of the 12 sets of lead signals or a combination thereof.

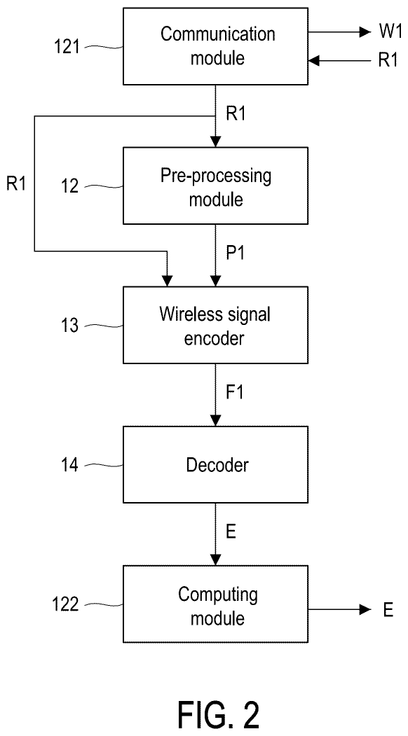

FIG. 2 is a schematic diagram illustrating estimation of an ECG signal E through wireless signal measurement according to an embodiment of the disclosure. The communication module 121 may transmit a wireless signal W1 through the transceiver 131 and receive a reflected signal R1 corresponding to the wireless signal W1. The pre-processing module 12 may perform pre-processing on the reflected signal R1 to generate a processed signal P1. The pre-processing module 12 may capture a signal corresponding to a specific waveform of the ECG from the reflected signal R1 as the processed signal P1. The specific waveform may include a P wave, a Q wave, a R wave, a S wave, or a T wave.

In an embodiment, before the reflected signal R1 is pre-processed, the pre-processing module 12 may first perform filtering processing on the reflected signal R1 to filter out signals related to the subject's breathing behavior in the reflected signal R1, so that the processed reflected signal R1 only retains information related to the micro-vibrations (i.e., the micro-vibrations associated with the heartbeat) of the subject's chest.

After the processed signal P1 is generated, the wireless signal encoder 13 may perform feature extraction on the processed signal P1 to capture an embedding F1 from the processed signal P1 and the reflected signal R1, where the embedding may also be referred to as a feature vector. Next, the decoder 14 may generate the estimated ECG signal E according to the embedding F1. The computing module 122 may output the estimated ECG signal E for user reference. For instance, the computing module 122 may output the estimated ECG signal E to a display that is communicatively connected to the detection device 10, so as to display the estimated ECG signal E through the display. Based on the above, the detection device 10 may estimate the ECG signal of the subject without using a contact sensor.

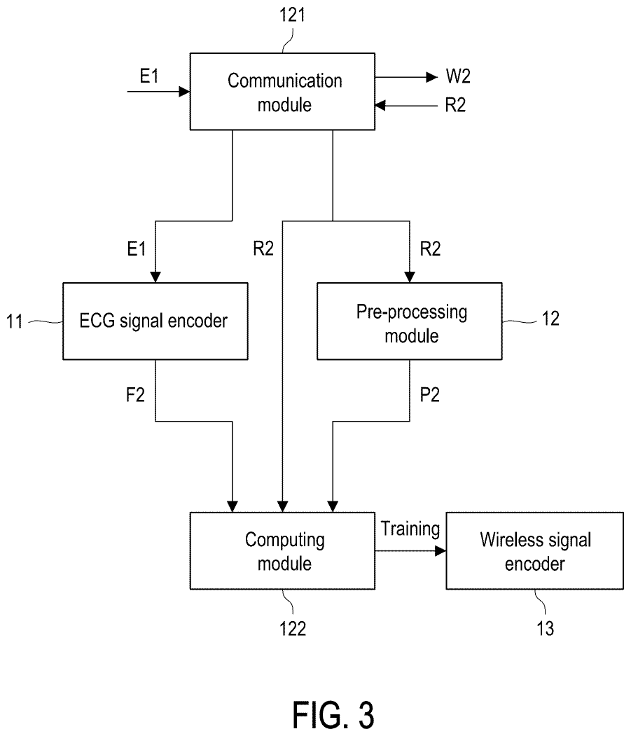

FIG. 3 is a schematic diagram illustrating training of the wireless signal encoder 13 according to an embodiment of the disclosure. The communication module 121 may detect an ECG signal E1 of the subject through the transceiver 132 and the electrodes. The ECG signal E1 is the real ECG signal of the subject. Further, the communication module 121 may transmit a wireless signal W2 to the subject through the transceiver 131 and receive a reflected signal R2 corresponding to the wireless signal W2. That is, the ECG signal E1 and the reflected signal R2 correspond to each other in the time domain.

The ECG signal encoder 11 may perform feature extraction on the ECG signal E1 to capture an embedding F2 from the ECG signal E1. The pre-processing module 12 may perform pre-processing on the reflected signal R2 to generate a processed signal P2. The computing module 122 may train the wireless signal encoder 13 according to the reflected signal R2 and the processed signal P2 based on a machine learning algorithm (e.g., a transformer algorithm). A loss function of the machine learning algorithm may be associated with the embedding F2. The wireless signal encoder 13 may be trained to output an embedding according to the input reflected signal and the processed signal, where the embedding output by the wireless signal encoder 13 will be similar to the embedding output by the ECG signal encoder 11.

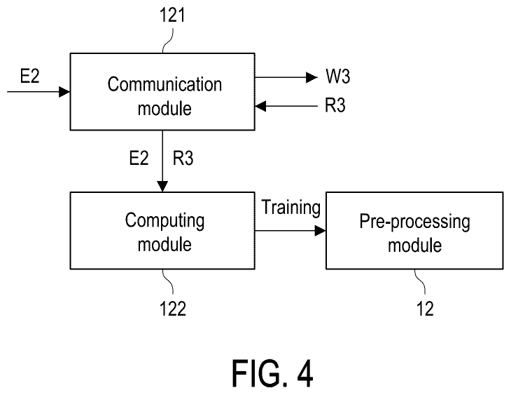

FIG. 4 is a schematic diagram illustrating training of the pre-processing module 12 according to an embodiment of the disclosure. The communication module 121 may detect an ECG signal E2 of the subject through the transceiver 132 and the electrodes. The ECG signal E2 is the real ECG signal of the subject. Further, the communication module 121 may transmit a wireless signal W3 to the subject through the transceiver 131 and receive a reflected signal R3 corresponding to the wireless signal W3. That is, the ECG signal E2 and the reflected signal R3 correspond to each other in the time domain.

The computing module 122 may train the pre-processing module 12 according to the reflected signal R3 based on a machine learning algorithm (e.g., a transformer algorithm). A loss function of the machine learning algorithm may be associated with the ECG signal E2. The pre-processing module 12 may be trained to output a processed signal according to the input reflected signal, and the processed signal output by the pre-processing module 12 will include a signal corresponding to a specific waveform of the ECG signal (i.e., the P wave, the Q wave, the R wave, the S wave, or the T wave).

To be specific, the computing module 122 may perform peak detection on the ECG signal E2 to detect a time when a specific waveform (i.e., the P wave, the Q wave, the R wave, the S wave, or the T wave) appears in the ECG signal E2. The reflected signal R3 may be used as a data point for label data, and the time associated with a specific waveform in the ECG signal E2 may be used as a label for the label data. The computing module 122 may train the pre-processing module 12 according to the label data. The trained pre-processing module 12 may output the time corresponding to a specific waveform (i.e., the P wave, the Q wave, the R wave, the S wave, or the T wave) according to the input reflected signal.

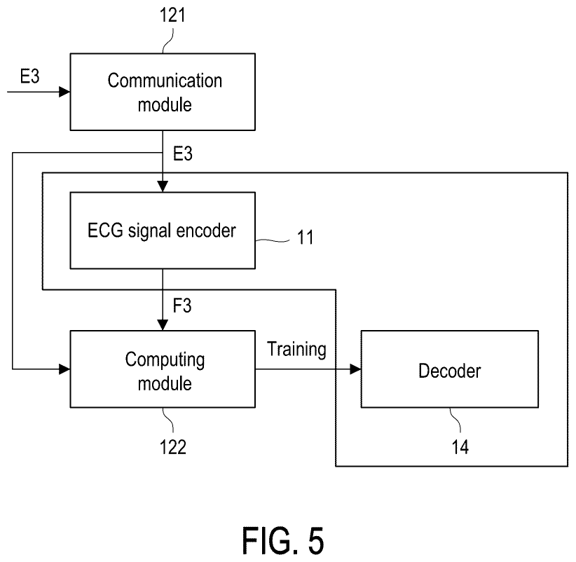

FIG. 5 is a schematic diagram illustrating training of the decoder 14 and the ECG signal encoder 11 according to an embodiment of the disclosure. The communication module 121 may detect an ECG signal E3 of the subject through the transceiver 132 and the electrodes. The ECG signal E3 is the real ECG signal of the subject. The ECG signal encoder 11 may perform feature extraction on the ECG signal E3 to capture an embedding F3 from the ECG signal E3. The computing module 122 may train or update the ECG signal encoder 11 and the decoder 14 according to the ECG signal E3 and the embedding F3 based on a machine learning algorithm (e.g., a transformer algorithm). A loss function of the machine learning algorithm may be associated with the ECG signal E3. The ECG signal encoder 11 and the decoder 14 may be trained, such that the decoder 14 may output an estimated ECG signal based on the embedding output by the ECG signal encoder 11. The estimated ECG signal will be close to the subject's real ECG signal (e.g., the input of the ECG signal encoder 11).

In an embodiment, the loss function of the machine learning algorithm used to train the decoder 14 may be associated with a spectrum of the ECG signal E3. For instance, the computing module 122 may perform short-time Fourier transform (STFT) on the ECG signal E3 to obtain the spectrum of the ECG signal E3. The loss function may be designed, so that the spectrum (e.g., the spectrum obtained by performing STFT on the estimated ECG signal by the computing module 122) of the estimated ECG signal output by the decoder 14 approximates the spectrum of the real ECG signal.

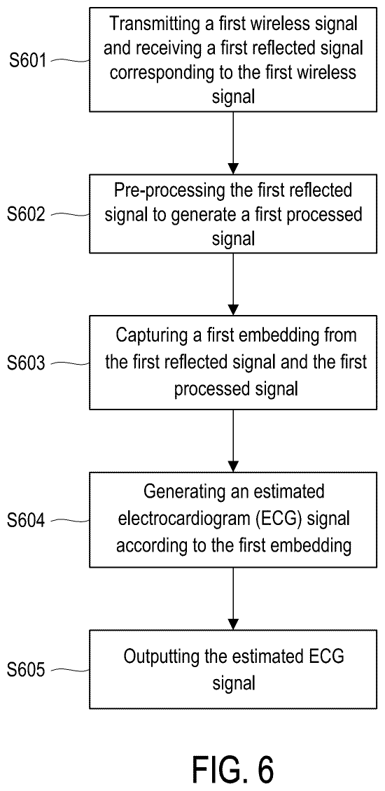

FIG. 6 is a flow chart illustrating a non-contact detection method for an ECG signal according to an embodiment of the disclosure, and the detection method may be implemented by the detection device 10 as shown in FIG. 1. In step S601, a first wireless signal is transmitted, and a first reflected signal corresponding to the first wireless signal is received. In step S602, the first reflected signal is pre-processed to generate a first processed signal. In step S603, a first embedding is captured from the first reflected signal and the first processed signal. In step S604, an estimated ECG signal is generated according to the first embedding. In step S605, the estimated ECG signal is output.

In view of the foregoing, in the disclosure, the detection device may measure the micro-vibrations of the human chest through the wireless signals to obtain the reflected signals and restore the reflected signals to the ECG signals based on the machine learning technology. Therefore, the detection device may obtain the ECG signal of the subject without using any wearable device or electrode patch. Some subjects may remove the ECG sensors from their bodies without permission because they dislike contact-type ECG sensors. In this case, when an emergency occurs or the subject's condition suddenly deteriorates, medical personnel may not be able to know the subject's condition in real time. Through the use of the detection device provided by the disclosure, the above situation may be avoided, the breadth of medical care is improved, and blind spots in medical care may be prevented.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A non-contact detection device for an electrocardiogram signal, comprising:
a first transceiver;
a storage medium storing a plurality of modules; and
a processor coupled to the storage medium and the first transceiver and accessing and executing the plurality of modules, wherein the plurality of modules comprise:
a communication module transmitting a first wireless signal through the first transceiver and receiving a first reflected signal corresponding to the first wireless signal;
a pre-processing module pre-processing the first reflected signal to generate a first processed signal;
a wireless signal encoder capturing a first embedding from the first reflected signal and the first processed signal;
a decoder generating an estimated electrocardiogram signal according to the first embedding; and
a computing module outputting the estimated electrocardiogram signal.

2. The detection device according to claim 1, further comprising:
a second transceiver coupled to the processor and detecting a first electrocardiogram signal, wherein the plurality of modules further comprise:
an electrocardiogram signal encoder capturing a second embedding from the first electrocardiogram signal, wherein
the communication module transmits a second wireless signal through the first transceiver and receives a second reflected signal corresponding to the second wireless signal,
the pre-processing module pre-processes the second reflected signal to generate a second processed signal, and
the computing module trains the wireless signal encoder according to the second reflected signal and the second processed signal based on a first machine learning algorithm, wherein a first loss function of the first machine learning algorithm is associated with the second embedding.

3. The detection device according to claim 2, wherein
the communication module transmits a third wireless signal through the first transceiver and receives a third reflected signal corresponding to the third wireless signal,
the communication module detects a second electrocardiogram signal through the second transceiver, and
the computing module trains the pre-processing module according to the third reflected signal based on a second machine learning algorithm, wherein a second loss function of the second machine learning algorithm is associated with the second electrocardiogram signal.

4. The detection device according to claim 3, wherein
the computing module detects a time when a waveform appears in the second electrocardiogram signal, wherein label data used to train the pre-processing module comprises the time.

5. The detection device according to claim 4, wherein the waveform comprises at least one of a P wave, a Q wave, a R wave, a S wave, and a T wave.

6. The detection device according to claim 1, further comprising:
a second transceiver coupled to the processor and detecting a third electrocardiogram signal, wherein the plurality of modules further comprise:
an electrocardiogram signal encoder capturing a third embedding from the third electrocardiogram signal, wherein
the computing module trains or updates the electrocardiogram signal encoder and the decoder according to the third electrocardiogram signal and the third embedding based on a third machine learning algorithm, wherein a third loss function of the third machine learning algorithm is associated with the third electrocardiogram signal.

7. The detection device according to claim 6, wherein the third loss function is associated with a spectrum of the third electrocardiogram signal.

8. The detection device according to claim 2, wherein at least one of the pre-processing module, the wireless signal encoder, the decoder, and the electrocardiogram signal encoder is a transformer model.

9. The detection device according to claim 1, wherein the first wireless signal comprises a frequency modulated continuous wave signal carried by millimeter waves.

10. A non-contact detection method for an electrocardiogram signal, comprising:
transmitting a first wireless signal and receiving a first reflected signal corresponding to the first wireless signal;
pre-processing the first reflected signal to generate a first processed signal;
capturing a first embedding from the first reflected signal and the first processed signal;
generating an estimated electrocardiogram signal according to the first embedding; and
outputting the estimated electrocardiogram signal.
